(19) United States
(12) Patent Application Publication
(10) Pub. No.: US 2026/0177523 A1
(43) Pub. Date: Jun. 25, 2026

(54) GRAPHENE-BASED FET DIAGNOSTIC SENSOR DEVICES

(71) Applicant: IPLEXMED LDA, Braga (PT)

(72) Inventors: Bruno Almeida, Porto (PT); Joana Rafaela Guerreiro, Porto (PT); Neide Vieira, Porto (PT)

(21) Appl. No.: 18/999,686

(22) Filed: Dec. 23, 2024

(51) Int. Cl. G01N 27/414; C12Q 1/6825

(52) U.S. Cl. CPC G01N 27/4145; C12Q 1/6825

(57) ABSTRACT

This disclosure generally relates to graphene-based diagnostic sensor devices. More particularly, the disclosure relates to graphene-based diagnostic sensor devices for the multiplex detection of target nucleic acids in fluid samples, including biological fluid samples. In certain aspects, the graphene-based multiplex diagnostic devices disclosed herein comprise a graphene-based FET multiplex sensor array having multiple independent sensors, each having a nucleic acid probe, for hybridizing and detecting a target nucleic acid, attached to a graphene monolayer.

### CROSS REFERENCE TO RELATED APPLICATIONS

N/A

### FIELD OF THE INVENTION

The present technology relates in general to graphene-based FET sensor array devices for the point-of-care multiplex detection of multiple infectious agents and antimicrobial resistance. More particularly, the present technology relates to graphene-based FET sensor array devices for the multiplex detection of target nucleic acids in fluid samples, including biological fluid samples, derived from multiple infectious agents and antimicrobial resistance genes.

### BACKGROUND

Many of the deadliest infectious diseases in the world can be prevented through timely diagnosis and treatment. However, current diagnostic tools have several limitations, including an inability to rapidly and accurately detect the presence or absence of multiple infectious agents simultaneously, efficiently, and at the point-of-care.

Therefore, alternative diagnostic tools are urgently needed, and the present disclosure provides graphene-based diagnostic sensor devices for the multiplex point-of-care detection of multiple target nucleic acids in biological samples.

Limitations and disadvantages of traditional sensors will become apparent to one of skill in the art, through comparison of such devices with certain aspects of the devices set forth in the remainder of this disclosure, including with reference to the claims.

### BRIEF SUMMARY

The present disclosure generally relates to graphene-based FET sensor array devices for the simultaneous point-of-care multiplex detection of multiple target nucleic acids, including nucleic acids derived from infectious agents (and optionally genes related to the development of antimicrobial resistance). In one aspect, the graphene-based FET sensor array devices have a sealable housing, an inlet configured to receive a biological fluid, a fluidic system for sample processing, and a sensor array within the sealable housing that is communicatively coupled to the inlet to receive the biological fluid.

In a still further aspect, the sensor array has multiple independent sensors. In this aspect, each sensor comprises: a graphene based field-effect transistor comprising a graphene monolayer; a nucleic acid probe for hybridizing and detecting a target nucleic acid; and at least one blocking agent. Still further, in this aspect, the graphene monolayer is functionalized with a linker for immobilizing the nucleic acid probe to the graphene monolayer, and the nucleic acid probe is terminally functionalized to bind to the linker. In another embodiment of this aspect, the nucleic acid probe is a pyrene modified nucleic acid probe, which does not require a linker for immobilization to the graphene monolayer. In this embodiment, the pyrene modified nucleic acid probe is directly immobilized on the graphene surface (i.e., utilizing π-π stacking directly on graphene,) The nucleic acid probe can also be modified with similar aromatic compounds (e.g., other polycyclic aromatic hydrocarbon having fused benzene rings, resulting in a flat aromatic system).

### BRIEF DESCRIPTION OF THE DRAWINGS

FIG. 1 illustrates a top view of an example graphene-based FET sensor array, in accordance with various example implementations of this disclosure.

### DETAILED DESCRIPTION

While the present technology will be described in connection with one or more preferred embodiments, it will be understood by those skilled in the art that the technology is not limited to only those particular embodiments. To the contrary, the presently described technology includes all alternatives, modifications, and equivalents as may be included within the spirit and scope of the appended claims.

Unless defined otherwise, all technical and scientific terms used herein have the same meaning as commonly understood by one of ordinary skill in the art to which the present disclosure belongs.

It must be noted that as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Generally, the present disclosure is directed to devices incorporating a graphene-based FET sensor array for the multiplex detection of multiple target nucleic acids in fluid samples. In one aspect, the graphene-based FET sensor array (or sensor array chip) is coupled to a sample collector that also enables sample pre-treatment. Each independent sensor of the graphene-based FET sensor array comprises a sensor having a graphene monolayer, and a unique nucleic acid probe bound to the graphene monolayer.

The graphene-based FET sensor arrays disclosed herein comprise multiple independent sensors, wherein each sensor independently comprises: a graphene field-effect transistor comprising a graphene monolayer; a nucleic acid probe for hybridizing and detecting a target nucleic acid; and at least one blocking agent. The graphene monolayer can be functionalized with a linker for immobilizing the nucleic acid probe directly to the graphene monolayer. In this aspect, the nucleic acid probe is terminally functionalized to bind to the linker.

In an alternative aspect, the nucleic acid probe is a polycyclic aromatic hydrocarbon (PAH) modified nucleic acid probe (e.g., a pyrene modified nucleic acid probe). In this aspect, the graphene monolayer is not functionalized with a linker for immobilizing the nucleic acid probe. Rather, the PAH modified nucleic acid probe binds to or is otherwise immobilized to the graphene surface, via π-π stacking (i.e., pi-pi stacking).

The PAH modified nucleic acid probe can be modified with a variety of PAH compounds, including for example: pyrene; naphthalene; biphenyl; fluorine; anthracene; phenanthrene; phenalene; tetracene; chrysene; triphenylene; pentacene; perylene; benzo[a]pyrene; corannulene; benzo[ghi]perylene; coronene; ovalene; or benzo[c]fluorene.

In one aspect, the nucleic acid probe is a 5′-end polycyclic aromatic hydrocarbon (PAH) modified nucleic acid probe. For example, in one embodiment the probe is a 5′-end pyrene modified nucleic acid probe in accordance with the following structure:

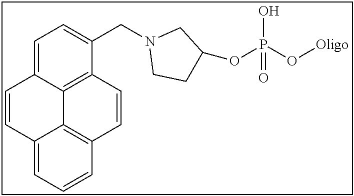

In another embodiment, the probe is a 5-end pyrene modified nucleic acid probe in accordance with the following structure:

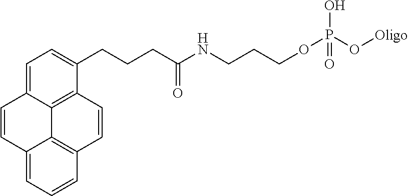

In other aspects, a poly-T (or other nucleotide) spacer sequence can be added before the target sequence to promote an upright conformation (relative to the graphene monolayer). In this aspect, the poly-T (or other nucleotide) spacer can be 3 to 6, 3 to 9, 3 to 12, 5 to 10, or 5 to 15 nucleotides in length.

The graphene-based FET sensor array devices disclosed herein are for the multiplex detection of target nucleic acids in biological or non-biological fluid or non-fluid samples. The target nucleic acid can be obtained from biological or non-biological fluid or non-fluid samples using methods known in the art. The target nucleic acid refers to a nucleic acid of interest, and can be deoxyribonucleic acid (DNA) or ribonucleic acid (RNA).

The nucleic acid probes used herein are generally single stranded synthetic deoxyribonucleic acid, and are complementary to target nucleic acids of interest. In alternative embodiments, the probes can comprise artificial nucleic acids, including for example: peptide nucleic acids; morpholino nucleic acids; locked nucleic acids, glycol nucleic acids; threose nucleic acid, and combinations thereof.

As used herein, the term “complementary” does not require that a sequence (e.g., a nucleic acid probe) is complementary over the full-length of a complementary strand (e.g., the target nucleic acid of interest), and can encompasses a sequence that is complementary to a portion of another sequence.

For example, a nucleic acid probe sequences can be complementary to a target nucleic acid sequence over a length ranging from about 2 to about 100 consecutive (contiguous) nucleotides, or any integer between 2 and 100. In some embodiments, the nucleic acid probe can be complementary to the target nucleic acid other over a length ranging from about 15 to about 30 consecutive (contiguous) nucleotides, or any integer between 15 and 30.

The nucleic acid probes used herein are complementary to target nucleic acids of interest such that the nucleic acids probes are capable of hybridizing (i.e., capable of forming a stable double stranded duplex via Watson-Crick base-pairing) with target nucleic acids. Despite some amount of mismatches, nucleic acid probe sequences generally have the ability to selectively hybridize to the designated target nucleic acid under appropriate conditions such as, for example, stringent and highly stringent conditions, such as those generally known by those of ordinary skill in the art.

Probes that can be used in combination with the graphene-based multiplex diagnostic devices disclosed herein, include any one or combination of probes set forth in U.S. Patent Application No. 63/671,309, the disclosure of which is hereby expressly incorporated by reference in its entirety and is hereby expressly made a portion of this application.

The target nucleic acid refers to a nucleic acid of interest, and can be deoxyribonucleic acid (DNA) or ribonucleic acid (RNA), and can be double stranded or single stranded. Nucleic acids of interest include nucleic acids derived from infectious agents, including, for example, viral or bacterial infectious agents. In some embodiments, the target nucleic acid may comprise mRNA, or cDNA wherein target DNA is created using isolated transcripts from a biological sample (i.e., wherein mRNA is reverse transcribed into using conventional techniques).

Target nucleic acids derived from bacterial sources include nucleic acids derived from, for example, Streptococcus pneumoniae, Staphylococcus aureus, Pseudomonas aeruginosa, Klebsiella pneumoniae, Haemophilus influenzae, Haemophilus parainfluenzae, Stenotrophomonas maltophilia, Chlamydophila pneumoniae, Moraxella catarrhalis, Achromobacter, Serratia marcescens, Escherichia coli, Acinetobacter, Enterobacter cloacae, Proteus mirabilis, Mycobacterium tuberculosis, Mycobacterium avium, Mycobacterium abscessus, Mycobacterium kansasii, and Mycobacterium gordonae.

Target nucleic acids derived from viral sources include, for example, nucleic acids derived from either DNA viruses or RNA viruses. DNA viruses include, for example, Adenoviruses (e.g., Human adenoviruses (types 3, 4, and 7)), Herpesviruses (e.g., Herpes simplex, varicella zoster, Epstein-Barr virus, cytomegalovirus, and Kaposi's sarcoma), Poxviruses (e.g., Vaccinia virus), Parvoviruses (e.g., Human parvovirus), Papovaviruses (e.g., Papilloma virus), and Hepadnaviruses (e.g., Hepatitis B virus). RNA viruses include, for example, Orthomyxoviruses (e.g., Influenza virus), Paramyxoviruses (e.g., Mumps, measles, respiratory syncytial virus), Coronaviruses (e.g., common cold viruses), Picornaviruses (e.g., Polio, coxsackie, hepatitis A, and rhinovirus), Reoviruses (e.g., Rotavirus, and reovirus), Togaviruses (e.g., Rubella, and arthropod-borne encephalitis), Flaviviruses (e.g., arthropod-borne viruses (yellow fever, dengue fever), Arenaviruses (e.g., Lymphocytic choriomeningitis, and Lassa fever), Rhabdoviruses (e.g., Rabies), and Retroviruses (e.g., Human T-cell leukemia virus, and HIV).

Target nucleic acids derived from fungal sources include, for example, nucleic acid derived from Candida, Blastomyces, Coccidioides, Cryptococcus, Histoplasma, Paracoccidioides, Aspergillus, Mucormycetes, Pneumocyctis, Taloromyces, and Sporothrix.

Target nucleic acids derived from protozoal sources include, for example, nucleic acid derived from Sarcodina (e.g., Entamoeba), Mastigophora (e.g., Giardia, and Leishmania), Ciliophora (e.g., Balantidium), and Sporozoa (e.g., Plasmodium, and Cryptosporidium).

Target nucleic acids can also be derived from genes conferring antimicrobial drug resistance, including, for example, extended-spectrum β-lactamases, CTX-M beta-lactamases, Carbapenemases, Klebsiella pneumoniae carbapenemase, New Delhi metallo beta lactamase, OXA-48-like carbapenemases, Verona integron-encoded metallo-β-lactamase, IMP-type metallo-β-lactamase, and methicillin resistance.

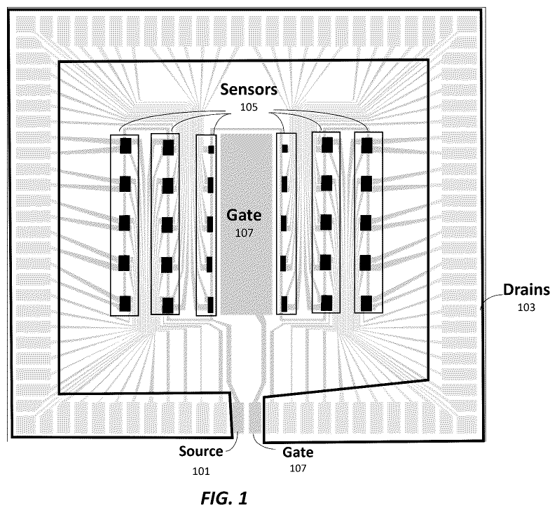

In one aspect of the disclosure, devices are provided having a graphene-based FET sensor array, wherein each sensor in the sensor array comprises a separate and independent sensor. Each separate and independent sensor further comprises a graphene monolayer, and a unique nucleic acid probe bound to the graphene monolayer. FIG. 1 illustrates a top view of an example graphene-based FET sensor array, in accordance with various example implementations of this disclosure. In this example, the terminals of the graphene-based FET sensor array comprise a common source terminal 101, a common gate terminal 107 and 98 drain terminals 103. Each of the 98 drain terminals 103 is connected to a graphene sensor 105.

In the embodiment illustrated in FIG. 1, the sensors 105 are in 20 groups of 4 sensors, 8 groups of 2 sensors, and 2 individual sensors. This particular grouping of 98 sensors 105 is an example design, and a smaller or larger number of sensors is also within the scope of this disclosure. For example, the sensor array can comprise from 60 to 120 sensors, from 70 to 120 sensors, from 80 to 120, from 90 to 120 sensors, from 60 to 100 sensors, from 70 to 100 sensors, from 80 to 100, or from 90 to 100 sensors. Further, the response of each sensor 105 may be measured by the corresponding drain 103, and a reader may read all sensors 105 simultaneously, or in groups.

Each sensor 105 comprises a graphene field-effect transistor comprising a graphene monolayer, which can be functionalized with a linker for immobilizing a unique nucleic acid probe (hybridizing to, and facilitating detection of, a unique target nucleic acid). The linker bonded, or otherwise attached to the graphene monolayer can be, for example, 1-pyrenebutyric acid succinimidyl ester, fluorenylmethyl succinimidyl carbonate, acridine orange succinimidyl ester, maleimide-PEG-amine, pyrene-PEG-amine, pyrene-PEG-maleimine or combinations thereof.

In alternative embodiments, each sensor 105 comprises a graphene field-effect transistor comprising a graphene monolayer, and a polycyclic aromatic hydrocarbon (PAH) modified nucleic acid probe (e.g., a pyrene modified nucleic acid probe). In this embodiment, the graphene monolayer is not functionalized with a linker for immobilizing the nucleic acid probe. Rather, the PAH modified nucleic acid probe binds to, or is otherwise immobilized to, the graphene surface, via π-π stacking.

Each sensor is also coated with at least one blocking agent. The blocking agent can be for blocking the graphene monolayer, for blocking the linker, or a combination of both. The blocking agent for blocking the graphene monolayer can be, for example, methoxypolyethylene glycol pyrene of different molecular weights, Tween 20, polyvinyl alcohol, or combinations thereof. The blocking agent for blocking the linker can be, for example, methoxypolyethylene glycol amine of different molecular weights, ethylamine, heptylamine, dodecylamine, or combinations thereof.

Each sensor also comprises a unique nucleic acid probe for hybridizing and detecting a unique target nucleic acid. The nucleic acid probe is generally a synthetic deoxyribonucleic acid, which can be terminally functionalized to bind to a linker bonded or attached to the graphene monolayer. The nucleic acid probe can be, for example, a synthetic deoxyribonucleic acid terminally functionalized with an amine functional group. The nucleic acid probe can also be, for example, a synthetic deoxyribonucleic acid terminally functionalized with a thiol functional group.

In other embodiments, the nucleic acid probe can be terminally functionalized with a polycyclic aromatic hydrocarbon (PAH) (e.g., pyrene). In this instance, the polycyclic aromatic hydrocarbon modified probe directly binds to the graphene surface, via π-π stacking.

In one aspect, there are disclosed diagnostic devices for the simultaneous detection of multiple target nucleic acids. These devices comprise graphene-based FET multiplex sensor arrays having multiple independent sensors (each sensor having a unique nucleic acid probe immobilized on a graphene monolayer) for the independent and simultaneous detection of target nucleic acids derived from multiple infectious agents.

For example, in one aspect, the graphene-based FET multiplex sensor array comprises multiple independent sensors for the simultaneous detection of target nucleic acids derived from Streptococcus pneumoniae, Staphylococcus aureus, Pseudomonas aeruginosa, Klebsiella pneumoniae, Haemophilus influenzae, Haemophilus parainfluenzae, Stenotrophomonas maltophilia, Chlamydophila pneumoniae, Moraxella catarrhalis, Achromobacter, Serratia marcescens, Escherichia coli, Acinetobacter, Enterobacter cloacae, Proteus mirabilis, Mycobacterium tuberculosis, Mycobacterium avium, Mycobacterium abscessus, Mycobacterium kansasii, Mycobacterium gordonae, or combinations thereof.

In yet another aspect, the graphene-based FET multiplex sensor array comprises multiple independent sensors for the simultaneous detection of target nucleic acids derived from genes conferring antimicrobial drug resistance, including, for example, extended-spectrum β-lactamases, CTX-M beta-lactamases, Carbapenemases, Klebsiella pneumoniae carbapenemase, New Delhi metallo beta lactamase, OXA-48-like carbapenemases, Verona integron-encoded metallo-β-lactamase, IMP-type metallo-β-lactamase, and methicillin resistance.

In at least one aspect, the graphene-based FET multiplex sensor array of the diagnostic device disclosed herein comprises multiple independent sensors, each having a graphene monolayer functionalized with 1-pyrenebutyric acid succinimidyl ester for the immobilization of a nucleic acid probe for the detection of a target nucleic acid of interest, and coated with a blocking agent derived from methoxypolyethylene glycol pyrene of different molecular weights, Tween 20, polyvinyl alcohol, or combinations thereof. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In at least one aspect, the graphene-based FET multiplex sensor array of the diagnostic device disclosed herein comprises multiple independent sensors, each having a graphene monolayer functionalized with 1-pyrenebutyric acid succinimidyl ester for the immobilization of a nucleic acid probe for the detection of a target nucleic acid of interest, and coated with a blocking agent derived from methoxypolyethylene glycol. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In at least one aspect, the graphene-based FET multiplex sensor array of the diagnostic device disclosed herein comprises multiple independent sensors, each having a graphene monolayer functionalized with 1-pyrenebutyric acid succinimidyl ester for the immobilization of a nucleic acid probe for the detection of a target nucleic acid of interest, and coated with a blocking agent derived Tween 20. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In at least one aspect, the graphene-based FET multiplex sensor array of the diagnostic device disclosed herein comprises multiple independent sensors, each having a graphene monolayer functionalized with 1-pyrenebutyric acid succinimidyl ester for the immobilization of a nucleic acid probe for the detection of a target nucleic acid of interest, and coated with a blocking agent derived polyvinyl alcohol. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In another aspect, the graphene-based FET multiplex sensor array of the diagnostic device disclosed herein comprises multiple independent sensors, each having a graphene monolayer functionalized with fluorenylmethyl succinimidyl carbonate for the immobilization of a nucleic acid probe for the detection of a target nucleic acid of interest, and coated with a blocking agent derived from methoxypolyethylene glycol pyrene, Tween 20, polyvinyl alcohol, or combinations thereof. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In another aspect, the graphene-based FET multiplex sensor array of the diagnostic device disclosed herein comprises multiple independent sensors, each having a graphene monolayer functionalized with fluorenylmethyl succinimidyl carbonate for the immobilization of a nucleic acid probe for the detection of a target nucleic acid of interest, and coated with a blocking agent derived from methoxypolyethylene glycol pyrene. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In another aspect, the graphene-based FET multiplex sensor array of the diagnostic device disclosed herein comprises multiple independent sensors, each having a graphene monolayer functionalized with fluorenylmethyl succinimidyl carbonate for the immobilization of a nucleic acid probe for the detection of a target nucleic acid of interest, and coated with a blocking agent derived from Tween 20. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In another aspect, the graphene-based FET multiplex sensor array of the diagnostic device disclosed herein comprises multiple independent sensors, each having a graphene monolayer functionalized with fluorenylmethyl succinimidyl carbonate for the immobilization of a nucleic acid probe for the detection of a target nucleic acid of interest, and coated with a blocking agent derived from polyvinyl alcohol. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In yet another aspect, the graphene-based FET multiplex sensor array of the diagnostic device disclosed herein comprises multiple independent sensors, each having a graphene monolayer functionalized with acridine orange succinimidyl ester for the immobilization of a nucleic acid probe for the detection of a target nucleic acid of interest, and also coated with a blocking agent derived from methoxypolyethylene glycol pyrene, Tween 20, polyvinyl alcohol, or combinations thereof. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In yet another aspect, the graphene-based FET multiplex sensor array of the diagnostic device disclosed herein comprises multiple independent sensors, each having a graphene monolayer functionalized with acridine orange succinimidyl ester for the immobilization of a nucleic acid probe for the detection of a target nucleic acid of interest, and also coated with a blocking agent derived from methoxypolyethylene glycol pyrene. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In yet another aspect, the graphene-based FET multiplex sensor array of the diagnostic device disclosed herein comprises multiple independent sensors, each having a graphene monolayer functionalized with acridine orange succinimidyl ester for the immobilization of a nucleic acid probe for the detection of a target nucleic acid of interest, and also coated with a blocking agent derived from Tween 20. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In yet another aspect, the graphene-based FET multiplex sensor array of the diagnostic device disclosed herein comprises multiple independent sensors, each having a graphene monolayer functionalized with acridine orange succinimidyl ester for the immobilization of a nucleic acid probe for the detection of a target nucleic acid of interest, and also coated with a blocking agent derived from polyvinyl alcohol. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In a still further aspect, the graphene-based FET multiplex sensor array of the diagnostic device disclosed herein comprises multiple independent sensors, each having a graphene monolayer functionalized with maleimide-PEG-amine for the immobilization of a nucleic acid probe, and also coated with a blocking agent derived from methoxypolyethylene glycol pyrene, Tween 20, polyvinyl alcohol, or combinations thereof. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In a still further aspect, the graphene-based FET multiplex sensor array of the diagnostic device disclosed herein comprises multiple independent sensors, each having a graphene monolayer functionalized with maleimide-PEG-amine for the immobilization of a nucleic acid probe, and also coated with a blocking agent derived from methoxypolyethylene glycol pyrene. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In a still further aspect, the graphene-based FET multiplex sensor array of the diagnostic device disclosed herein comprises multiple independent sensors, each having a graphene monolayer functionalized with maleimide-PEG-amine for the immobilization of a nucleic acid probe, and also coated with a blocking agent derived from Tween 20. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In a still further aspect, the graphene-based FET multiplex sensor array of the diagnostic device disclosed herein comprises multiple independent sensors, each having a graphene monolayer functionalized with maleimide-PEG-amine for the immobilization of a nucleic acid probe, and also coated with a blocking agent derived from polyvinyl alcohol. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In a another aspect, the graphene-based FET multiplex sensor array of the diagnostic device disclosed herein comprises multiple independent sensors, each having a graphene monolayer functionalized with pyrene-PEG-amine for the immobilization of a nucleic acid probe, and also coated with a blocking agent derived from methoxypolyethylene glycol pyrene, Tween 20, polyvinyl alcohol, or combinations thereof. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In a another aspect, the graphene-based FET multiplex sensor array of the diagnostic device disclosed herein comprises multiple independent sensors, each having a graphene monolayer functionalized with pyrene-PEG-amine for the immobilization of a nucleic acid probe, and also coated with a blocking agent derived from methoxypolyethylene glycol pyrene. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In a another aspect, the graphene-based FET multiplex sensor array of the diagnostic device disclosed herein comprises multiple independent sensors, each having a graphene monolayer functionalized with pyrene-PEG-amine for the immobilization of a nucleic acid probe, and also coated with a blocking agent derived Tween 20. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

In a another aspect, the graphene-based FET multiplex sensor array of the diagnostic device disclosed herein comprises multiple independent sensors, each having a graphene monolayer functionalized with pyrene-PEG-amine for the immobilization of a nucleic acid probe, and also coated with a blocking agent derived from polyvinyl alcohol. In certain alternative embodiments of this aspect, the graphene monolayer is non-functionalized and a polycyclic aromatic hydrocarbon (PAH) modified nucleic acid probe is used, wherein the probe is immobilized directly to the graphene monolayer via π-π stacking.

### ITEMS OF THE INVENTION

The invention further relates to the following items:
1. A graphene-based multiplex diagnostic device comprising: a sealable housing; an inlet configured to receive a biological fluid; a graphene-based FET sensor array within the sealable housing and communicatively coupled to the inlet to receive the biological fluid; wherein the sensor array comprises multiple independent sensors, each sensor comprising: a graphene field-effect transistor comprising a graphene monolayer; a nucleic acid probe for hybridizing and detecting a target nucleic acid; and at least one blocking agent; wherein the graphene monolayer is functionalized with a linker for immobilizing the nucleic acid probe to the graphene monolayer; and wherein the nucleic acid probe is terminally functionalized to bind to the linker.
2. The diagnostic device of item 1, wherein the nucleic acid probe is a synthetic deoxyribonucleic acid.
3. The diagnostic device of item 1, wherein the nucleic acid probe is a synthetic deoxyribonucleic acid terminally functionalized with an amine functional group.
4. The diagnostic device of item 1, wherein the nucleic acid probe is a synthetic deoxyribonucleic acid terminally functionalized with a thiol functional group.
5. The diagnostic device of item 1, wherein the linker is selected from the group consisting of 1-pyrenebutyric acid succinimidyl ester, fluorenylmethyl succinimidyl carbonate, acridine orange succinimidyl ester, maleimide-PEG-amine, pyrene-PEG-amine, pyrene-PEG-maleimine and combinations thereof.
6. The diagnostic device of item 1, wherein the blocking agent is for blocking the graphene monolayer and selected from the group consisting of methoxypolyethylene glycol pyrene of diverse molecular weights, Tween 20, polyvinyl alcohol, and combinations thereof.
7. The diagnostic device of item 1, wherein the blocking agent is for blocking the linker and selected from the group consisting of methoxypolyethylene glycol amine of diverse molecular weights, ethylamine, heptylamine, dodecylamine, and combinations thereof.
8. The diagnostic device of item 1, wherein the linker is 1-pyrenebutyric acid succinimidyl ester and the blocking agent is methoxypolyethylene glycol pyrene of diverse molecular weights.
9. The diagnostic device of item 1, wherein the linker is 1-pyrenebutyric acid succinimidyl ester and the blocking agent is Tween 20.
10. The diagnostic device of item 1, wherein the linker is 1-pyrenebutyric acid succinimidyl ester and the blocking agent is polyvinyl alcohol.
11. The diagnostic device of item 1, wherein the linker is fluorenylmethyl succinimidyl carbonate and the blocking agent is methoxypolyethylene glycol pyrene of diverse molecular weights.
12. The diagnostic device of item 1, wherein the linker is fluorenylmethyl succinimidyl carbonate and the blocking agent is Tween 20.
13. The diagnostic device of item 1, wherein the linker is fluorenylmethyl succinimidyl carbonate and the blocking agent is polyvinyl alcohol.
14. The diagnostic device of item 1, wherein the linker is acridine orange succinimidyl ester and the blocking agent is methoxypolyethylene glycol pyrene of diverse molecular weights.
15. The diagnostic device of item 1, wherein the linker is acridine orange succinimidyl ester and the blocking agent is Tween 20.
16. The diagnostic device of item 1, wherein the linker is acridine orange succinimidyl ester and the blocking agent is polyvinyl alcohol.
17. The diagnostic device of item 1, wherein the linker is maleimide-PEG-amine and the blocking agent is methoxypolyethylene glycol pyrene of diverse molecular weights.
18. The diagnostic device of item 1, wherein the linker is maleimide-PEG-amine and the blocking agent is Tween 20.
19. The diagnostic device of item 1, wherein the linker is maleimide-PEG-amine and the blocking agent is polyvinyl alcohol.
20. The diagnostic device of item 1, wherein the linker is pyrene-PEG-amine and the blocking agent is methoxypolyethylene glycol pyrene of diverse molecular weights.
21. The diagnostic device of item 1, wherein the linker is pyrene-PEG-amine and the blocking agent is Tween 20.
22. The diagnostic device of item 1, wherein the linker is pyrene-PEG-amine and the blocking agent is polyvinyl alcohol.
23. The diagnostic device of any one of items 1-22, wherein the nucleic acid probe hybridizes to a target nucleic acid derived from an infectious agent.
24. The diagnostic device of any one of items 1-22, wherein the graphene-based FET multiplex sensor array comprises independent sensors for the simultaneous detection of between 20 to 30 different target nucleic acids.
25. The diagnostic device of any one of items 1-22, wherein the graphene-based FET multiplex sensor array comprises independent sensors for the simultaneous detection of target nucleic acids derived from an infectious agent, a gene related to the development of antimicrobial resistance, or a combination thereof.
26. The diagnostic device of item 25, wherein the infectious agent is viral, bacterial, protozoan, or fungal.
27. The diagnostic device of item 26, wherein the viral infectious agent is a DNA virus or RNA virus.
28. The diagnostic device of item 27, wherein the DNA virus is an Adenovirus, a Herpesvirus, a Poxvirus, a Parvovirus, a Hepadnavirus, a Papovaviruse or a combination thereof.
29. The diagnostic device of item 27, wherein the RNA virus is an Orthomyxovirus, a Paramyxovirus, a Picornavirus, a Reovirus, a Togavirus, a Flavivirus, an Arenavirus, a Retrovirus, a Coronaviruse, a Rhabdoviruse, or combinations thereof.
30. The diagnostic device of item 26, wherein the bacterial infectious agent is Streptococcus, Staphylococcus, Pseudomonas, Klebsiella, Haemophilus, Stenotrophomonas, Chlamydophila, Moraxella, Achromobacter, Serratia, Escherichia, Acinetobacter, Enterobacter, Proteus, Mycobacterium, or a combination thereof.
31. The diagnostic device of item 26, wherein the fungal infectious agent is Candida, Blastomyces, Coccidioides, Cryptococcus, Histoplasma, Paracoccidioides, Aspergillus, Mucormycetes, Pneumocyctis, Taloromyces, Sporothrix, or a combination thereof.
32. The diagnostic device of item 26, wherein the protozoan infectious agent is Sarcodina, Mastigophora, Ciliophora, Sporozoa, or a combination thereof.
33. The diagnostic device of item 25, wherein the gene related to the development of antimicrobial resistance is an extended-spectrum β-lactamase, a CTX-M beta-lactamase, a Carbapenemase, a Klebsiella pneumoniae carbapenemase, a New Delhi metallo beta lactamase, a OXA-48-like carbapenemase, a Verona integron-encoded metallo-β-lactamase, an IMP-type metallo-β-lactamase, methicillin or a combination thereof.
34. A graphene-based multiplex diagnostic device comprising: a sealable housing; an inlet configured to receive a biological fluid; a graphene-based FET sensor array within the sealable housing and communicatively coupled to the inlet to receive the biological fluid; wherein the sensor array comprises multiple independent sensors, each sensor comprising: a graphene field-effect transistor comprising a graphene monolayer; a nucleic acid probe for hybridizing and detecting a target nucleic acid; and at least one blocking agent; wherein the nucleic acid probe is a polycyclic aromatic hydrocarbon modified nucleic acid probe.
35. The diagnostic device of item 34, wherein the polycyclic aromatic hydrocarbon modified nucleic acid probe binds directly to the graphene monolayer.
36. The diagnostic device of item 35, wherein the polycyclic aromatic hydrocarbon modified nucleic acid probe binds directly to the graphene monolayer via pi-pi stacking.
37. The diagnostic device of item 34, wherein the polycyclic aromatic hydrocarbon is pyrene.
38. The diagnostic device of item 34, wherein the nucleic acid probe is a synthetic deoxyribonucleic acid.
39. The diagnostic device of item 34, wherein the blocking agent is selected from the group consisting of methoxypolyethylene glycol pyrene of diverse molecular weights, Tween 20, polyvinyl alcohol, and combinations thereof.
40. The diagnostic device of item 34, wherein the graphene-based FET multiplex sensor array comprises independent sensors for the simultaneous detection of target nucleic acids derived from an infectious agent, a gene related to the development of antimicrobial resistance, or a combination thereof.
41. The diagnostic device of item 40, wherein the infectious agent is viral, bacterial, protozoan, or fungal.
42. The diagnostic device of item 41, wherein the viral infectious agent is a DNA virus or RNA virus.
43. The diagnostic device of item 42, wherein the DNA virus is an Adenovirus, a Herpesvirus, a Poxvirus, a Parvovirus, a Hepadnavirus, a Papovaviruse or a combination thereof.
44. The diagnostic device of item 42, wherein the RNA virus is an Orthomyxovirus, a Paramyxovirus, a Picornavirus, a Reovirus, a Togavirus, a Flavivirus, an Arenavirus, a Retrovirus, a Coronaviruse, a Rhabdoviruse, or combinations thereof.
45. The diagnostic device of item 41, wherein the bacterial infectious agent is Streptococcus, Staphylococcus, Pseudomonas, Klebsiella, Haemophilus, Stenotrophomonas, Chlamydophila, Moraxella, Achromobacter, Serratia, Escherichia, Acinetobacter, Enterobacter, Proteus, Mycobacterium, or a combination thereof.
46. The diagnostic device of item 41, wherein the fungal infectious agent is Candida, Blastomyces, Coccidioides, Cryptococcus, Histoplasma, Paracoccidioides, Aspergillus, Mucormycetes, Pneumocyctis, Taloromyces, Sporothrix, or a combination thereof.
47. The diagnostic device of item 41, wherein the protozoan infectious agent is Sarcodina, Mastigophora, Ciliophora, Sporozoa, or a combination thereof.
48. The diagnostic device of item 40, wherein the gene related to the development of antimicrobial resistance is an extended-spectrum β-lactamase, a CTX-M beta-lactamase, a Carbapenemase, a Klebsiella pneumoniae carbapenemase, a New Delhi metallo beta lactamase, a OXA-48-like carbapenemase, a Verona integron-encoded metallo-β-lactamase, an IMP-type metallo-β-lactamase, methicillin or a combination thereof.

The present invention should not be considered limited to the particular examples described above. Various modifications, equivalent processes, as well as numerous structures to which the present disclosure may be applicable, and which fall within the general scope of the disclosure, will be readily apparent to those of skill in the art to which the present disclosure is directed upon review of the instant specification.

What is more, as will be apparent to those of skill in the art upon reading this disclosure, each of the individual aspects described and illustrated herein has discrete components and features which may be readily separated from or combined with the features of any of the other several aspects, without departing from the scope or spirit of the present disclosure.

What is claimed is:

1. A graphene-based multiplex diagnostic device comprising:
a sealable housing;
an inlet configured to receive a biological fluid;
a graphene-based FET sensor array within the sealable housing and communicatively coupled to the inlet to receive the biological fluid;
wherein the sensor array comprises multiple independent sensors, each sensor comprising:
a graphene field-effect transistor comprising a graphene monolayer;
a nucleic acid probe for hybridizing and detecting a target nucleic acid; and
at least one blocking agent;
wherein the graphene monolayer is functionalized with a linker for immobilizing the nucleic acid probe to the graphene monolayer; and
wherein the nucleic acid probe is terminally functionalized to bind to the linker.

2. The diagnostic device of claim 1, wherein the nucleic acid probe is a synthetic deoxyribonucleic acid terminally functionalized with an amine or thiol functional group.

3. The diagnostic device of claim 1, wherein the linker is selected from the group consisting of 1-pyrenebutyric acid succinimidyl ester, fluorenylmethyl succinimidyl carbonate, acridine orange succinimidyl ester, maleimide-PEG-amine, pyrene-PEG-amine, pyrene-PEG-maleimine and combinations thereof.

4. The diagnostic device of claim 1, wherein the blocking agent is for blocking the graphene monolayer and selected from the group consisting of methoxypolyethylene glycol pyrene of diverse molecular weights, Tween 20, polyvinyl alcohol, and combinations thereof.

5. The diagnostic device of claim 1, wherein the blocking agent is for blocking the linker and selected from the group consisting of methoxypolyethylene glycol amine of diverse molecular weights, ethylamine, heptylamine, dodecylamine, and combinations thereof.

6. The diagnostic device of claim 1, wherein the graphene-based FET multiplex sensor array comprises independent sensors for the simultaneous detection of between 20 to 30 different target nucleic acids.

7. The diagnostic device of claim 1, wherein the graphene-based FET multiplex sensor array comprises independent sensors for the simultaneous detection of target nucleic acids derived from an infectious agent, a gene related to the development of antimicrobial resistance, or a combination thereof.

8. The diagnostic device of claim 7, wherein the infectious agent is a DNA virus, RNA virus, bacterial, protozoan, or fungal.

9. The diagnostic device of claim 8, wherein the DNA virus is an Adenovirus, a Herpesvirus, a Poxvirus, a Parvovirus, a Hepadnavirus, a Papovaviruse or a combination thereof, and the RNA virus is an Orthomyxovirus, a Paramyxovirus, a Picornavirus, a Reovirus, a Togavirus, a Flavivirus, an Arenavirus, a Retrovirus, a Coronaviruse, a Rhabdoviruse, or combinations thereof.

10. The diagnostic device of claim 8, wherein the bacterial infectious agent is Streptococcus, Staphylococcus, Pseudomonas, Klebsiella, Haemophilus, Stenotrophomonas, Chlamydophila, Moraxella, Achromobacter, Serratia, Escherichia, Acinetobacter, Enterobacter, Proteus, Mycobacterium, or a combination thereof.

11. The diagnostic device of claim 7, wherein the gene related to the development of antimicrobial resistance is an extended-spectrum β-lactamase, a CTX-M beta-lactamase, a Carbapenemase, a Klebsiella pneumoniae carbapenemase, a New Delhi metallo beta lactamase, a OXA-48-like carbapenemase, a Verona integron-encoded metallo-β-lactamase, an IMP-type metallo-β-lactamase, methicillin or a combination thereof.

12. A graphene-based multiplex diagnostic device comprising:
a sealable housing;
an inlet configured to receive a biological fluid;
a graphene-based FET sensor array within the sealable housing and communicatively coupled to the inlet to receive the biological fluid;
wherein the sensor array comprises multiple independent sensors, each sensor comprising:
a graphene field-effect transistor comprising a graphene monolayer;
a nucleic acid probe for hybridizing and detecting a target nucleic acid; and
at least one blocking agent;
wherein the nucleic acid probe is a polycyclic aromatic hydrocarbon modified nucleic acid probe.

13. The diagnostic device of claim 12, wherein the polycyclic aromatic hydrocarbon modified nucleic acid probe binds directly to the graphene monolayer via pi-pi stacking.

14. The diagnostic device of claim 12, wherein the polycyclic aromatic hydrocarbon is pyrene.

15. The diagnostic device of claim 12, wherein the blocking agent is selected from the group consisting of methoxypolyethylene glycol pyrene of diverse molecular weights, Tween 20, polyvinyl alcohol, and combinations thereof.

16. The diagnostic device of claim 12, wherein the graphene-based FET multiplex sensor array comprises independent sensors for the simultaneous detection of target nucleic acids derived from an infectious agent, a gene related to the development of antimicrobial resistance, or a combination thereof.

17. The diagnostic device of claim 16, wherein the infectious agent is a DNA virus, RNA virus, bacterial, protozoan, or fungal.

18. The diagnostic device of claim 17, wherein the DNA virus is an Adenovirus, a Herpesvirus, a Poxvirus, a Parvovirus, a Hepadnavirus, a Papovaviruse or a combination thereof, and the RNA virus is an Orthomyxovirus, a Paramyxovirus, a Picornavirus, a Reovirus, a Togavirus, a Flavivirus, an Arenavirus, a Retrovirus, a Coronaviruse, a Rhabdoviruse, or combinations thereof.

19. The diagnostic device of claim 17, wherein the bacterial infectious agent is Streptococcus, Staphylococcus, Pseudomonas, Klebsiella, Haemophilus, Stenotrophomonas, Chlamydophila, Moraxella, Achromobacter, Serratia, Escherichia, Acinetobacter, Enterobacter, Proteus, Mycobacterium, or a combination thereof, and the fungal infectious agent is Candida, Blastomyces, Coccidioides, Cryptococcus, Histoplasma, Paracoccidioides, Aspergillus, Mucormycetes, Pneumocyctis, Taloromyces, Sporothrix, or a combination thereof, and the protozoan infectious agent is Sarcodina, Mastigophora, Ciliophora, Sporozoa, or a combination thereof.

20. The diagnostic device of claim 16, wherein the gene related to the development of antimicrobial resistance is an extended-spectrum β-lactamase, a CTX-M beta-lactamase, a Carbapenemase, a Klebsiella pneumoniae carbapenemase, a New Delhi metallo beta lactamase, a OXA-48-like carbapenemase, a Verona integron-encoded metallo-β-lactamase, an IMP-type metallo-β-lactamase, methicillin or a combination thereof.
